(19) United States
(12) Patent Application Publication
(10) Pub. No.: US 2014/0302161 A1
(43) Pub. Date: Oct. 9, 2014

(54) VASELINE SUBSTITUTE

(71) Applicant: BEIERSDORF AG, Hamburg (DE)

(72) Inventors: Anja Bauer, Hamburg (DE); Luis Arturo Carbajal Avila, Berlin (DE)

(21) Appl. No.: 14/357,794

(22) PCT Filed: Nov. 8, 2012
(86) PCT No.: PCT/EP2012/072110

§ 371 Date: May 13, 2014

(30) Foreign Application Priority Data
Nov. 14, 2011 (DE) 102011086261.7

(51) Int. Cl. A61K 8/97; A61Q 19/00; A61K 8/92

(52) U.S. Cl. CPC A61K 8/97; A61K 8/927; A61Q 19/00
USPC 424/539

(57) ABSTRACT

Lipid mixture of a) shea butter and b) beeswax in A weight ratio of shea butter/beeswax of from 3:1 to 15:1.

The present invention relates to a lipid mixture of shea butter and beeswax, the use thereof and use in cosmetic preparations.

Vaseline is a colloidal system of branched-chain solid hydrocarbons and high-boiling straight-chain liquid hydrocarbons which forms a semisolid viscous mass which has a melting point of about 38-56° C. and is virtually insoluble in water, alcohol and glycerol. It was developed/discovered in 1859 by the American chemist Robert Chesebrough in Pennsylvania and is used in cosmetics and pharmacy as a salve or cream base. Moreover, it is used in many products of daily life as glidant, lubricant etc. Vaseline (also called petrolatum) is obtained from petroleum and is per se well tolerated by the human body. However, being a petroleum product, it is not a “renewable raw material”.

In cosmetics, there has for many years been a trend toward “green” products. An increasing number of consumers wants their cosmetic cleansing and care products to be not only well tolerated and sensorially appealing, but moreover also produced from renewable, generally plant-based raw materials which are not only biodegradable but can also be produced in a near-natural and environmentally compatible manner Being a petroleum product, Vaseline does not fall under these raw materials.

On the other hand, Vaseline is not only a cost-effective cosmetic ingredient. Its good pharmaceutical and sensory properties, good compatibility with other cosmetic ingredients, as well as the skin, make Vaseline a virtually indispensible constituent of cosmetic care products.

It was therefore the object of the present invention to develop a substitute for Vaseline which, on the one hand, consists of plant-based raw materials that can be produced in an environmentally friendly manner and which, on the other hand, has the same galenic and sensory properties and compatibilities as Vaseline.

Surprisingly, the object is achieved by a lipid mixture of

a) shea butter and

b) beeswax

in the weight ratio of shea butter/beeswax of 3:1 to 15:1.

According to the invention, it is advantageous if the shea butter contained in the lipid mixture according to the invention is obtained from Butyrospermum parkii.

Moreover, it is advantageous according to the invention if the shea butter has a melting range of 34 to 46° C. under standard conditions. According to the invention, a pressure of 1.013 bar is considered to be a standard condition.

It is preferred according to the invention if the saponification number of the shea butter is 175-185 mg KOH/g.

Embodiments of the present invention preferred according to the invention are also characterized in that the acid number of the shea butter is less than/equal to 0.5 mg KOH/g.

Furthermore, it is preferred according to the invention if the iodine number of the shea butter is 64-77 g iodine/100 g.

Furthermore, it is preferred according to the invention if the peroxide number of the shea butter is less than/equal to 5.4.

Last but not least, a shea butter preferred according to the invention is characterized in that the unsaponifiable fraction is 8-13.4%.

Lipid mixtures advantageous according to the invention are characterized in that Cera Alba is used as beeswax.

Beeswax preferred according to the invention is characterized by the fact that the dropping point is 60.5-65.4° C.

Beeswax preferred according to the invention has an acid number of 16.5-24.4 mg KOH/g.

It is preferred according to the invention if the ester number of the beeswax is 69.5-80.4 mg KOH/g.

Last but not least, it is preferred according to the invention if the saponification number of the beeswax is 86.5-104.4 mg KOH/g.

Lipid mixtures preferred according to the invention are characterized in that the weight ratio of shea butter to beeswax is from 4.5:1 to 12:1.

According to the invention, the lipid mixture according to the invention can advantageously be incorporated into cosmetic preparations.

According to the invention, a cosmetic preparation comprising a lipid mixture according to the invention in a concentration of from 1 to 80% by weight, based on the total weight of the preparation, is advantageous. According to the invention, the lipid mixture is preferably used in a concentration of from 10 to 70% by weight, based on the total weight of the preparation.

It is advantageous according to the invention if the water content of the preparation is less than 0.5% by weight, based on the total weight of the preparation.

Moreover, according to the invention, the preparations according to the invention can advantageously comprise dyes, perfume substances and/or aroma substances, it being possible to use all dyes, perfume substances and aroma substances known for lip cosmetics for this purpose.

Embodiments of the cosmetic preparation according to the invention that are advantageous according to the invention are characterized in that the mixture or preparation is free from Vaseline, paraffins and mineral oil.

Embodiments of the cosmetic preparation according to the invention that are advantageous according to the invention are also characterized in that the preparation is free from polyethylene glycols and derivatives thereof.

It is also advantageous according to the invention if the cosmetic preparation is free from parabens.

Embodiments of the present invention that are advantageous according to the invention are also characterized in that the cosmetic preparation is free from silicone oils or waxes.

It is advantageous according to the invention if the cosmetic preparation according to the invention comprises one or more antioxidants.

Antioxidants preferred according to the invention are here considered to be BHT (butylated hydroxytoluene), tocopherol and tocopherol acetate.

Furthermore, the cosmetic preparation according to the invention advantageously comprises further plant or animal oils, fats and/or waxes.

The use of the lipid mixture according to the invention as substitute for Vaseline in cosmetic preparations is in accordance with the invention.

### EXAMPLES

The examples below serve to illustrate the present invention without limiting it. The data always refer to % by weight unless stated otherwise.

| | Common | | | | | | |
|---|---|---|---|---|---|---|---|
| INCI | name | 1 | 2 | 3 | 4 | 5 | 6 |
| Cera Alba | Beeswax | 5 | 5 | 4.87 | 4.87 | 5 | 10 |
| Butyrospermum | Shea | | | 55.87 | 45.87 | 47.5 | 45 |
| Parkii Butter | Butter 1 | | | | | | |
| Butyrospermum | Shea | 10.285 | 12.57 | | | | |
| Parkii Butter | Butter 2 | | | | | | |
| Butyrospermum | Shea | 34.715 | 42.43 | | | | |
| Parkii Butter | Butter 3 | | | | | | |
| Cetyl Palmitate | Cetyl | | | 3.65 | 3.65 | | |
| | palmitate | | | | | | |
| Hydrogenated | Hydro- | | | 7.18 | 9.2 | | |
| Vegetable Oil | genated | | | | | | |
| | vegetable oil | | | | | | |
| Canola Oil | Canola oil | 40.91 | 31.82 | | | | |
| Caprylic/ | Caprylic/ | | | 18.38 | 23.54 | | |
| Capric | capric | | | | | | |
| Triglyceride | triglyceride | | | | | | |
| Simmondsia | Jojoba oil | | | 5.74 | 7.35 | 47.5 | 45 |
| Chinensis | | | | | | | |
| Seed Oil | | | | | | | |
| Olus Oil (and) | Olus oil and | 4.09 | 3.18 | | | | |
| Camelina | Camelina | | | | | | |
| Sativa Oil | Sativa oil | | | | | | |
| Prunus | Almond oil | | | 4.31 | 5.52 | | |
| Amygdalus | | | | | | | |
| Dulcis Oil | | | | | | | |
| Polyglyceryl-3- | Poly- | 5 | 5 | | | | |
| Polyricinoleate | glyceryl- | | | | | | |
| | 3-polyricin- | | | | | | |
| | oleate | | | | | | |

1.-13. (canceled)

14. A lipid mixture of shea butter and beeswax, wherein a weight ratio of shea butter to beeswax is from 3:1 to 15:1.

15. The lipid mixture of claim 14, wherein the shea butter has a melting range under standard conditions of from 34° C. to 46° C.

16. The lipid mixture of claim 14, wherein the beeswax comprises Cera Alba.

17. The lipid mixture of claim 14, wherein the weight ratio of shea butter to beeswax is from 4.5:1 to 12:1.

18. The lipid mixture of claim 14, wherein the mixture is free from Vaseline, paraffins and mineral oil.

19. The lipid mixture of claim 15, wherein the weight ratio of shea butter to beeswax is from 4.5:1 to 12:1, the beeswax comprises Cera Alba, and the mixture is free from Vaseline, paraffins and mineral oil.

20. A cosmetic composition, wherein the composition comprises the lipid mixture of claim 14 in a concentration of from 1% to 80% by weight, based on a total weight of the composition.

21. The composition of claim 20, wherein the composition has a water content of less than 0.5% by weight, based on a total weight of the composition.

22. The composition of claim 20, wherein the composition comprises aroma substances.

23. The composition of claim 20, wherein the composition is free from Vaseline, paraffins and mineral oil.

24. The composition of claim 20, wherein the composition is free from polyethylene glycols and derivatives thereof.

25. The composition of claim 20, wherein the composition is free from parabens.

26. The composition of claim 20, wherein the composition is free from silicone oils or waxes.

27. The composition of claim 20, wherein the composition further comprises one or more antioxidants.

28. The composition of claim 20, wherein the the shea butter has a melting range under standard conditions of from 34° C. to 46° C.

29. The composition of claim 20, wherein the weight ratio of shea butter to beeswax is from 4.5:1 to 12:1.

30. The composition of claim 29, wherein the composition has a water content of less than 0.5% by weight, based on a total weight of the composition.

31. The composition of claim 30, wherein the composition is free from silicone oils or waxes.

32. The composition of claim 30, wherein the composition further comprises one or more antioxidants.

33. A method of replacing Vaseline in a cosmetic composition, wherein the method comprises incorporating in the composition the lipid mixture of claim 14 instead of Vaseline.
